(19) United States
(12) Patent Application Publication
(10) Pub. No.: US 2008/0044894 A1
(43) Pub. Date: Feb. 21, 2008

(54) Cellular culture chip device

(75) Inventors: Gwo-Bin Lee, Tainan City (TW); Song-Bin Huang, Taipei Hsien (TW)

Correspondence Address: BIRCH STEWART KOLASCH & BIRCH, PO BOX 747, FALLS CHURCH, VA, 22040-0747, US

(73) Assignee: National Cheng Kung University

(21) Appl. No.: 11/797,052

(22) Filed: Apr. 30, 2007

(30) Foreign Application Priority Data
Aug. 15, 2006 (TW) 095129916

(51) Int. Cl. C12M 1/00

(52) U.S. Cl. 435/307.1

(57) ABSTRACT

A cellular culture chip device includes a chip body. The chip body includes: a first flow channel having a first inlet end and a first outlet end and adapted to direct a liquid nutrient; a second flow channel adapted to direct a gellable culture medium; a medium retaining hole fluidly connected to the second flow channel and adapted to retain the gellable culture medium, the medium retaining hole having an opening connected fluidly to the first flow channel and adapted to expose the gellable culture medium to the liquid nutrient flowing through the first flow channel; a pump membrane adapted to control the flow of the liquid nutrient within the first flow channel; and a pressure channel unit to operate the pump membrane.

### CROSS-REFERENCE TO RELATED APPLICATION

This application claims priority of Taiwanese application no. 095129916, filed on Aug. 15, 2006.

### BACKGROUND OF THE INVENTION

1. Field of the Invention

This invention relates to a chip device, more particularly to a cellular culture chip device.

2. Description of the Related Art

At present, cellular culture is usually performed by culturing cells in a medium plate containing a gellable medium coated on a bottom surface of the medium plate and a fluid medium over the gellable medium, or a medium plate containing only a fluid medium. During the culture process, the fluid medium should be replaced by a fresh fluid medium to provide the cultured cells sufficient nutrition and to remove metabolite produced by the cells. Although change of the medium is conducted in a disinfection environment, the cell culture is still likely to be contaminated due to the manual operation by an operator. In addition, refreshing the fluid medium or drawing the fluid medium from the medium plate for observing the cells in the gellable medium or on the medium plate is time and manpower wasting.

Therefore, there is a need in the art to provide a cellular culture chip device in which a fluid medium can be automatically drawn out from the cellular culture chip device.

### SUMMARY OF THE INVENTION

Therefore, the object of the present invention is to provide a cellular culture chip device that can overcome the aforesaid drawbacks of the prior art.

According to this invention, a cellular culture chip device includes a chip body having: a first flow channel having a first inlet end and a first outlet end and adapted to direct a liquid nutrient; a second flow channel adapted to direct a gellable culture medium; a medium retaining hole fluidly connected to the second flow channel and adapted to retain the gellable culture medium, the medium retaining hole having an opening connected fluidly to the first flow channel and adapted to expose the gellable culture medium to the liquid nutrient flowing through the first flow channel; a pump membrane adapted to control the flow of the liquid nutrient within the first flow channel; and a pressure channel unit to operate the pump membrane.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is an exploded perspective view of the first preferred embodiment of a cellular culture chip device according to this invention;

FIG. 2 is a plan view illustrating the state where a second plate is stacked on a first plate in the first preferred embodiment;

FIG. 3 is a schematic cross-sectional view of the first preferred embodiment;

FIGS. 4(a) to 4(d) are schematic cross-sectional views illustrating consecutive steps for cell culture performed on the first preferred embodiment of the cellular culture chip device;

FIG. 5 is an exploded perspective view of the second preferred embodiment of a cellular culture chip device according to this invention;

FIG. 6 is a plan view of the second preferred embodiment; and

FIGS. 7(a) to 7(d) are schematic cross-sectional views illustrating consecutive steps for cell culture performed on the second preferred embodiment of the cellular culture chip device.

### DETAILED DESCRIPTION OF THE PREFERRED EMBODIMENTS

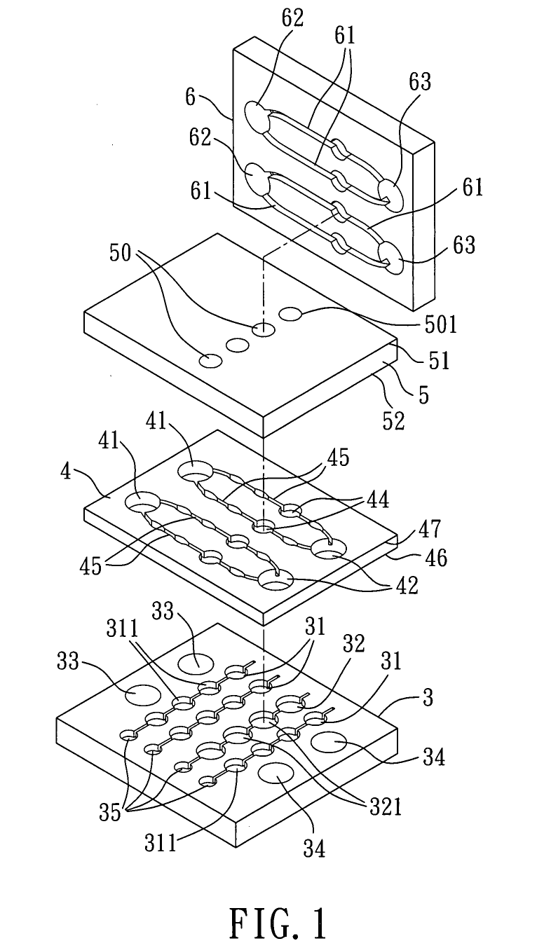

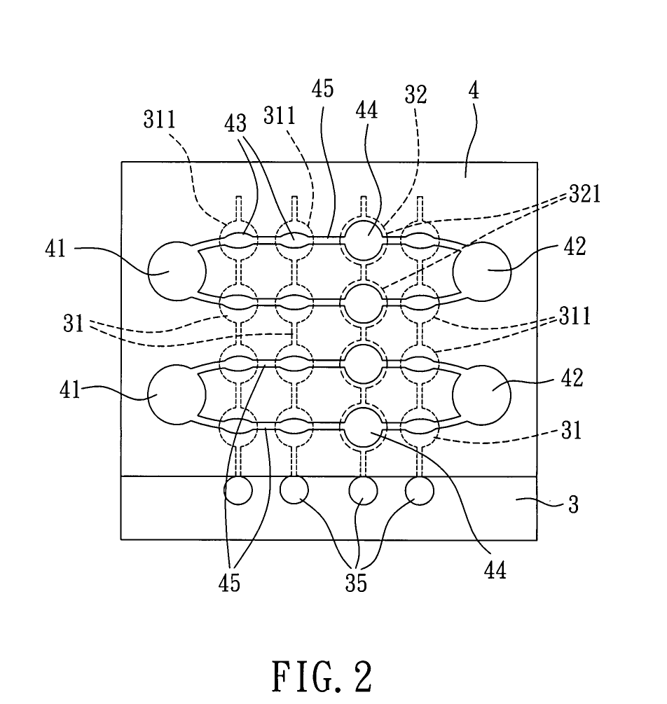

Referring to FIGS. 1, 2, and 3, the first preferred embodiment of a cellular culture chip device according to the present invention is shown to include a chip body formed of a first plate 3, a second plate 4, a third plate 5, and a fourth plate 6, which are stacked sequentially. In this embodiment, the first, second, third, and fourth plates 3, 4, 5, 6, are made of a polydimethylsiloxane (PDMS) material which is biocompatible and transparent. In this embodiment, the thickness of the second plate 4 is smaller than those of the first, third, and fourth plates 3, 5, 6. The thickness and the material used in the present invention should not be limited and can vary based on actual requirements.

The first plate 3 includes a pressure channel unit formed of three spaced apart pump pressure channels 31 and a valve pressure channel 32 disposed in parallel with the pump pressure channels 31 and between the right two pump pressure channels 31 (see FIG. 1). Each of the pump pressure channels 31 has four pressurizing sections 311, and the valve pressure channel 32 has four pressurizing sections 321. The first plate 3 further includes four pressure inlet/outlet holes 35 fluidly communicated with the pump and valve pressure channels 31, 32, respectively.

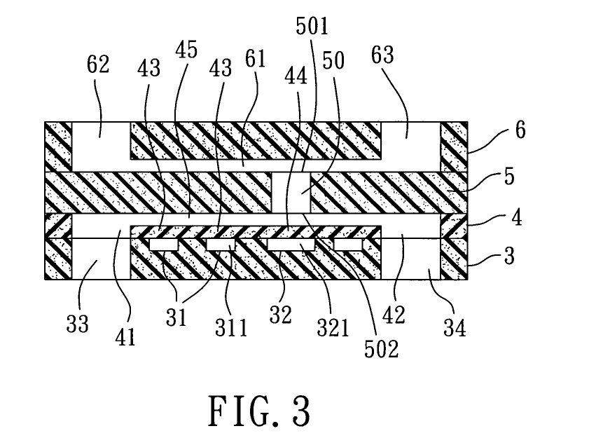

The second plate 4 is superposed over the first plate 3 and includes four first flow channels 45 extending in a direction that intersects the pump and valve pressure channels 31, 32, and adapted to direct a liquid nutrient. As the first flow channels 45 do not penetrate a bottom surface 46 of the second plate 4, the second plate 4 has pump membranes 43 (see FIG. 3) operated by the pump pressure channels 31, and valve membranes 44 operated by the valve pressure channel 32. Each of the pump membranes 43 is formed between the respective one of the pressurizing sections 311 in the pump pressure channels 31 and the respective one of the first flow channels 45, and is adapted to control the flow of the liquid nutrient within the first flow channels 45. Each of the valve membranes 44 is formed between the respective one of the pressurizing sections 321 in the valve pressure channel 32 and the respective one of the first flow channels 45. Each of the first and second plates 3, 4 has two first injection holes 33, 41, and two first drain holes 34, 42. One of the first injection holes 33 in the first plate 3 cooperates with the respective one of the first injection holes 41 in the second plate 4 to define a first inlet end fluidly communicated with two of the first flow channels 45. Similarly, one of the first drain holes 34 in the first plate 3 cooperates with the respective one of the first drain holes 42 in the second plate 4 to define a first outlet end fluidly communicated with the respective one of the first inlet ends through the respective two first flow channels 45.

The fourth plate 6 includes four second flow channels 61 adapted to direct a gellable culture medium and extending substantially in the same direction as the first flow channels 45, two second inlet ends 62, and two second outlet ends 63 each of which is fluidly communicated with the respective one of the second inlet ends 62 through the respective two second flow channels 61.

The third plate 5 is interposed between the second plate 4 and the fourth plate 6, and includes four medium retaining holes 50 extending through top and bottom surfaces 51, 52 of the third plate 5 and adapted to retain the gellable culture medium. Each of the medium retaining holes 50 is disposed over the respective one of the valve membranes 44, is fluidly connected to the respective one of the first and second flow channels 45, 61, and has a cross-section smaller than that of the respective one of the valve membranes 44. Each of the medium retaining holes 50 has an upper opening 501 in the top surface 51 of the third plate 5 and a lower opening 502 in the bottom surface 52 of the third plate 5 (see FIG. 3). The lower opening 502 of each of the medium retaining holes 50 can be blocked by the respective one of the valve membranes 44, and is adapted to expose the gellable culture medium retained therein to the liquid nutrient flowing through the respective one of the first flow channels 45.

In use, the first injection holes 33, 41 (cooperatively defining the first inlet ends) are connected to liquid nutrient storage tanks (not shown), and the first drain holes 34, 42 (cooperatively defining the first outlet ends) are connected to an external collection tube (not shown). The pressure inlet/outlet holes 35 are connected to an air compressor (not shown) for supplying or withdrawing a compressed gas to or from the pump and valve pressure channels 31, 32. The second inlet ends 62 are connected to gellable culture medium storage tanks (not shown), and the second outlet ends 63 are connected to an external collection tube (not shown).

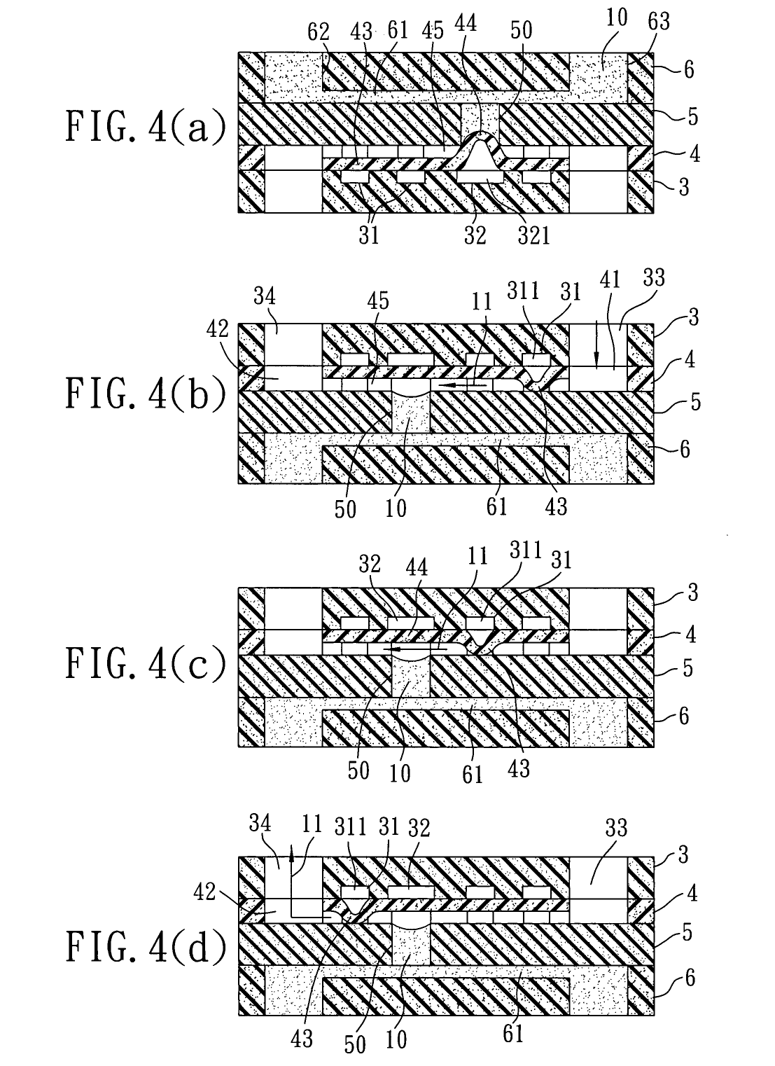

Consecutive operating steps for cell culture using the first preferred embodiment of the cellular culture chip device according to this invention are shown in FIGS. 4(a) to 4(d). As shown in FIG. 4(a), the valve pressure channel 32 is filled with the compressed gas such that each of the valve membranes 44 projects resiliently into the respective first flow channel 45 and is seated against the respective one of the lower openings 502 of the medium retaining holes 50. Thereafter, the gellable culture medium 10 containing cells suspended therein is introduced into the second flow channels 61 through the second inlet ends 62, and is filled in the medium retaining holes 50. The introduction of the gellable culture medium 10 may be done by suction using a vacuum pump connected to the second outlet ends 63. After the gellable culture medium 10 introduced in the second flow channels 61 and the medium retaining holes 50 is solidified to a gel state, the compressed gas is withdrawn from the valve pressure channel 32 so that the valve membranes 44 return to their original positions, and the cellular culture chip device is inverted so that the first plate 3 becomes the uppermost plate (see FIG. 4(b)).

As shown in FIGS. 4(b) to 4(d), the pump membranes 43 are sequentially and intermittently forced into the first flow channels 45 by injecting the compressed gas into the pump pressure channels 31 sequentially from the right to the left so as to produce a pumping effect. With the pumping effect, the fluid medium flows through the first flow channels 45 from the first injection holes 33, 41 to the first drain holes 34, 42 along a direction indicated, by arrow 11, and contacts an exposed surface of the solidified gellable culture medium 10 in the medium retaining holes 50 so as to supply nutrition to the cells in the solidified gellable culture medium 10. The injection of the compressed gas to the pump and valve pressure channels 31, 32 can be automatically operated, and the time of injection can be preset.

It should be noted that the numbers of the components, such as the first and second flow channels 45, 61, the pump and valve pressure channels 31, 32, and the pump and valve membranes 43, 44, may be varied as desired and should not be limited to those shown in the drawings.

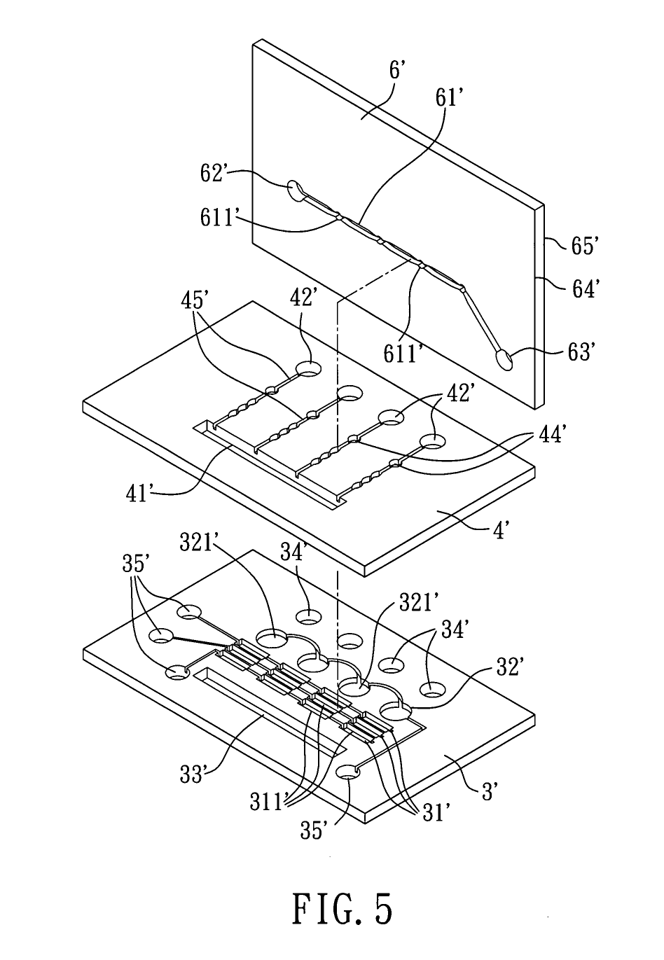

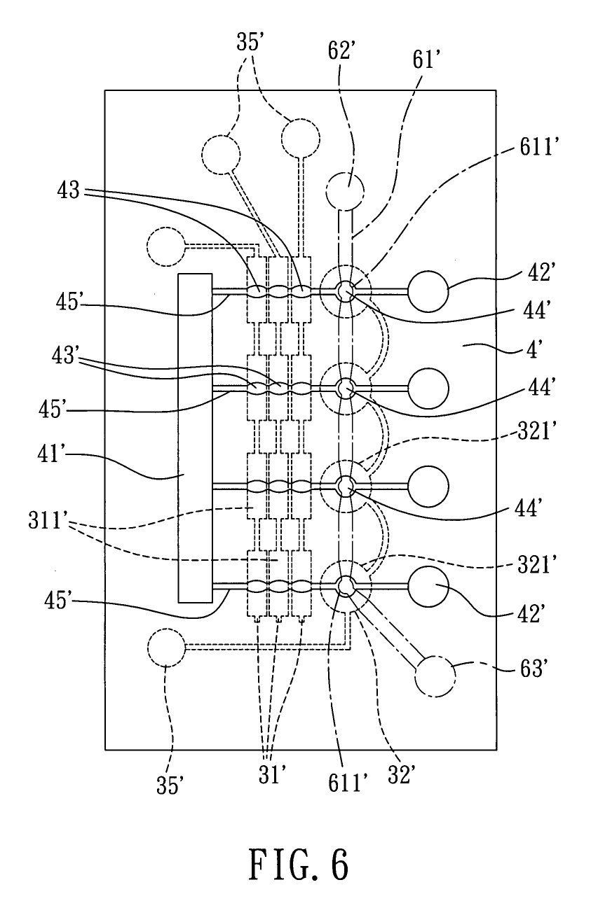

FIGS. 5, 6, and 7(a) to 7(d) illustrate the second preferred embodiment of the cellular culture chip device according to this invention. The cellular culture chip device includes a chip body formed of a first plate 3′, a second plate 4′, and a third plate 6′, which are stacked sequentially. The third plate 5 used in the first preferred embodiment is dispensed with in the second preferred embodiment.

The first plate 3′ includes three spaced apart pump pressure channels 31′ having four pressurizing sections 311′, a valve pressure channel 32′ disposed adjacent to the rightmost pump pressure channel 31′ and having four pressurizing sections 321′, a first injection hole 33′ in the form of a long slit, four first drain holes 34′, and four pressure inlet/outlet holes 35′.

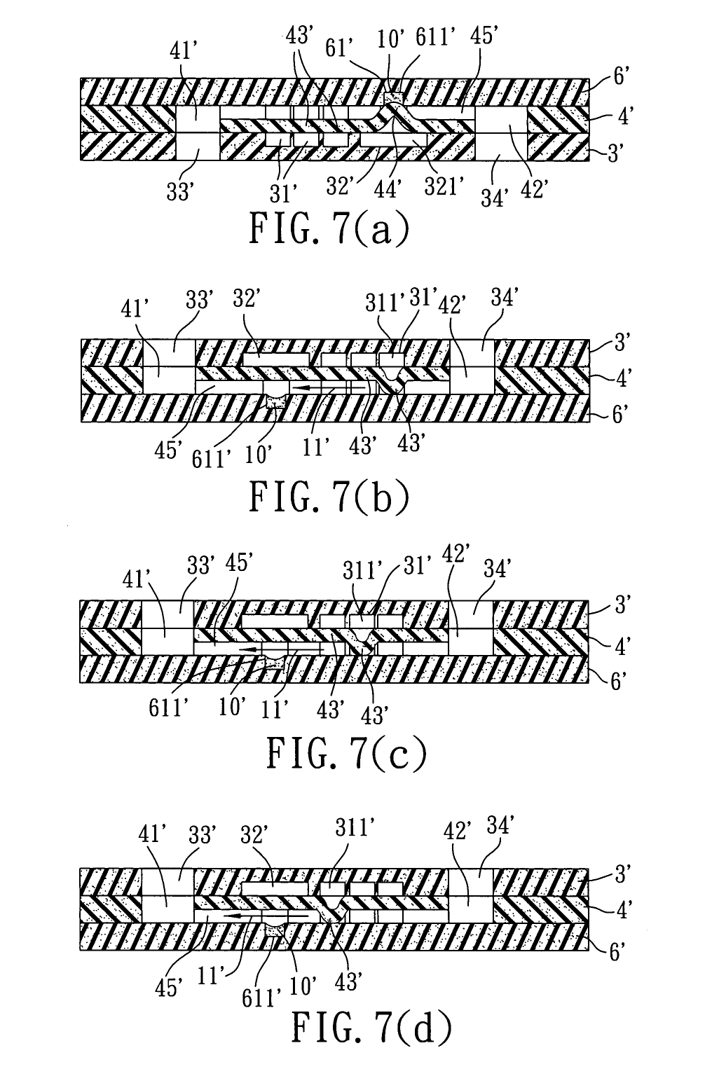

The second plate 4′ includes a first injection hole 41′ aligned with the first injection hole 33′ in the first plate 3′, four first drain holes 42′ respectively aligned with the first drain holes 34′ in the first plate 3′, four first flow channels 45′, pump membranes 43′ each of which is formed between the respective one of the pressurizing sections 311′ in the pump pressure channels 31′ and the respective one of the first flow channels 45′ (see FIGS. 7(a) to 7(d)), and valve membranes 44′ each of which is formed between the respective one of the pressurizing sections 321′ in the valve pressure channels 32′ and the respective one of the first flow channels 45′ (see FIGS. 7(a) to 7(d)).

The third plate 6′ includes a second flow channel 61′ intersecting the first flow channels 45′ in the second plate 4′ and having a second inlet end 62′ and a second outlet end 63′. Unlike the first preferred embodiment, the medium retaining holes 611′ in this embodiment are formed in the second flow channel 61′ at intersections of the first and second flow channels 45′, 61′. Each of the medium retaining holes 611′ extends through a bottom surface 64′ of the third plate 6′ so as to form an opening connected to the respective first flow channel 45′, but does not extend through the upper surface 65′ of the third plate 6′.

The process of using the cellular culture chip device of this preferred embodiment for cell culture is illustrated in FIGS. 7(a) to 7(d), and is substantially the same as that of the previous embodiment shown in FIGS. 4(a) to 4(d). Thus, the description thereof is omitted herein.

By virtue of the pump and valve pressure channels 31, 32, 31′, 32′, the pump and valve membranes 43, 44, 43′, 44′, and the medium retaining holes 50, 611′, the gellable culture medium 10 (containing cells) can be retained in the medium retaining holes 50, 611′, and the fluid medium can flow through the medium retaining holes 50, 611′ by the pumping effect produced by the pump membranes 43, 43′ and the pump pressure channels 31, 31′ so as to contact the gellable culture medium in the medium retaining holes 50, 611′. As the liquid nutrient can be introduced into and discharged automatically from the cellular culture chip device of the present invention, the liquid nutrient can be refreshed easily and automatically. Therefore, the contamination and time waste problems associated with the prior art can be avoided.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. A cellular culture chip device comprising:
a chip body which includes:
a first flow channel having a first inlet end and a first outlet end and adapted to direct a liquid nutrient;
a second flow channel adapted to direct a gellable culture medium;
a medium retaining hole fluidly connected to said second flow channel and adapted to retain the gellable culture medium, said medium retaining hole having an opening connected fluidly to said first flow channel and adapted to expose the gellable culture medium to the liquid nutrient flowing through said first flow channel;
a pump membrane adapted to control the flow of the liquid nutrient within said first flow channel; and
a pressure channel unit to operate said pump membrane.

2. The cellular culture chip device as claimed in claim 1, wherein said chip body further includes a valve membrane operated by said pressure channel unit to block said opening of said medium retaining hole.

3. The cellular culture chip device as claimed in claim 2, wherein said first flow channel is disposed above said pump and valve membranes, said second flow channel being disposed above said first flow channel, said medium retaining hole being disposed between said first and second flow channels, said valve membrane being operable to project resiliently into said first flow channel and to seat against said opening of said medium retaining hole.

4. The cellular culture chip device as claimed in claim 3, wherein said chip body is formed of first, second, third and fourth plates which are stacked sequentially, said pressure channel unit being formed in said first plate, said first flow channel being formed in said second plate, said pump and valve membranes being formed between said pressure channel unit and said first flow channel, said medium retaining hole being formed in said third plate, said second flow channel being formed in said fourth plate.

5. The cellular culture chip device as claimed in claim 4, wherein said pressure channel unit includes a pump pressure channel and a valve pressure channel which are formed in said first plate, said first flow channel extending in a direction that intersects said pump and valve pressure channels, said pump membrane being formed in said second plate and between said pump pressure channel and said first flow channel, said valve membrane being formed in said second plate and between said valve pressure channel and said first flow channel.

6. The cellular culture chip device as claimed in claim 4, wherein said first and second flow channels extend substantially in the same direction.

7. The cellular culture chip device as claimed in claim 4, wherein said first inlet and outlet ends are disposed in said first and second plates, said second flow channel having second inlet and outlet ends that are disposed in said fourth plate.

8. The cellular culture chip device as claimed in claim 2, wherein said first flow channel is disposed above said pump and valve membranes, said second flow channel being disposed above and intersecting said first flow channel, said medium retaining hole being disposed in said second flow channel at an intersection of said first and second flow channels, said valve membrane being operable to project resiliently into said first flow channel and to seat against said opening.

9. The cellular culture chip device as claimed in claim 8, wherein said chip body is formed of first, second, and third plates which are stacked sequentially, said pressure channel unit being formed in said first plate, said first flow channel being disposed in said second plate, said pump and valve membranes being disposed between said pressure channel unit and said first flow channel, said second flow channel and said medium retaining hole being disposed in said third plate.

10. The cellular culture chip device as claimed in claim 9, wherein said pressure channel unit includes a pump pressure channel and a valve pressure channel, said pump membrane being formed in said second plate and between said pump pressure channel and said first flow channel, said valve membrane being formed in said second plate and between said valve pressure channel and said first flow channel.

11. The cellular culture chip device as claimed in claim 9, wherein said first inlet and outlet ends are disposed in said first and second plates, said second flow channel having second inlet and outlet ends disposed in said third plate.

12. The cellular culture chip device as claimed in claim 2, wherein said chip body is made of a material which is transparent and biocompatible.
